(19) United States
(12) Patent Application Publication
(10) Pub. No.: US 2002/0028228 A1
(43) Pub. Date: Mar. 7, 2002

(54) BIOCONTROL FOR PLANTS WITH PAENIBACILLUS MACERANS, PSEUDOMONAS PUTIDA, AND SPOROBOLOMYCES ROSEUS

(76) Inventors: GARY C. BERGSTROM, ITHACA, NY (US); WILMAR CORIO DA LUZ, PASSO FUNDO (BR)

Correspondence Address: MICHAEL L GOLDMAN, ESQ., NIXON PEABODY LLP, CLINTON SQUARE P O BOX 31051, ROCHESTER, NY, 14603, US

(21) Appl. No.: 09/118,656

(22) Filed: Jul. 17, 1998

Related U.S. Application Data
(60) Provisional application No. 60/053,310, filed on Jul. 22, 1997.

(30) Foreign Application Priority Data
Jul. 17, 1998 (US) PCT/US98/15365

(51) Int. Cl. C12N 1/12; C12N 1/20; A01N 63/00; A01N 25/00; A01N 65/00; A01N 63/04

(52) U.S. Cl. 424/405; 424/93.3; 424/93.4; 424/93.47; 424/93.5

(57) ABSTRACT

The present invention is directed to isolated Paenibacillus macerans, Pseudomonas putida, and Sporoboloinyces roseus which are useful as a biocontrol agent. These organisms are useful in a method of imparting to plants protection against plant pathogens by applying them to plants, plant seeds, or soil surrounding plants under conditions effective to impart disease protection to the plants or plants produced from the plant seeds. The biocontrol agents are also useful in a method of enhancing plant growth which involves applying them to plants, plants seeds, or soil surrounding plants under conditions effective to enhance growth in the plants or plants produced from the plant seeds.

This application claims benefit of U.S. Provisional Patent Application Ser. No. 60/053,310, filed Jul. 22, 1997.

The present invention was made with support under USDA Hatch Project No. NYC153-472. The U.S. Government may have certain rights.

### FIELD OF THE INVENTION

The present invention relates to biocontrol for plants with Paenibacillus macerans, Pseudomonas putida, and Sporobolomyces roseus.

### BACKGROUND OF THE INVENTION

There are approximately 40 biocontrol products commercially available for the control of plant diseases worldwide. Biocontrol products are available to control many diverse pathogens, as recently reviewed by Fravel, et al., “Availability and Application of Biocontrol Products,” Biological and Culture Tests for Control of Plant Diseases, 11:1-7 (1996). At least 27 genera of fungi, 3 genera of bacteria, and 4 genera of nematodes are targeted for control by these products. More than half of these products control soilborne fungi. The biocontrol agents themselves are also diverse and include at least 9 genera of fungi, 4 genera of bacteria, and one actinomycete. Biocontrol products are used on a great variety of crops including greenhouse crops, row crops, field crops, perennial field crops, and trees and wood, as well as in special cropping systems such as mushroom cultivation. The products are applied in many ways. They may be sprayed onto plants or harvested fruits, drenched on harvested fruit or on plants, incorporated into the soil, applied as root dips, used to treat seeds, or inserted into trees or wood products. Biocontrol products currently on the market in the U.S. include Aspire, AQ-10, Galltrol A, Norbac 84C, Bio-Save 10, Bio-Save 11, Blightban A506, Victus, Epic, Kodiak, Deny, Mycostop, Binab-T and W, T-22G and T-22HB, and SoilGard.

Pathogens are controlled by biocontrol agents of the same species or genus as the pathogen in several cases. For example, nonpathogenic Agrobacterium radiobacter is used to control crown gall (Galltrol-A, Nogall, Diegall). Nonpathogenic Fusarium oxysporum is used to control F. oxysporum (Biofox C, Fusaclean) and F. moniliforme (Biofox C). Nonpathogenic Pseudomonas solanacearum controls pathogenic P. solanacearum (PSSOL), while P. fluorescens is used to control P. tolassii (Conquer, Victus). Pythium oligandrum is used to control P. ultimum (Polygandron). These agents may work through antibiosis (A. radiobacter; Kerr, A. “Biological Control of Crown Gall through Production of Agrocin 84,” Plant Dis., 64:25-30 (1980)), competition and induced systemic resistance (Fusaclean; Alabouvette, et al., “Recent Advances in the Biological Control of Fusarium Wilts,” Pestic. Sci., 37:365-373 (1993)), parasitism (Polygandron; Vesely, D. “Germinating Power of Oospores of Pythium oligandrum in a Powder Preparation,” Folia Microbiol., 32:502 (1987)).

Some biocontrol agents control only one pathogen. For example, Conquer and Victus both contain P. fluorescens used to control mushroom blotch caused by P. tolassii. Biocontrol agents are sometimes perceived as serving only niche markets since many products have narrow applicability. In part because of this perception, many biocontrol products are manufactured by small companies. However, most biocontrol agents have multiple pathogen and crop uses. For example, SoilGard controls damping-off incited by Rhizoctonia solani and Pythium spp. on bedding plants and vegetable transplants, as well as Sclerotium rolfsii on carrot and pepper in the field (Lumsden et al., “Biological Control of Damping-off Caused by Pythium ultimum and Rhizoctonia solani with Gliocladium virens in Soilless Mix,” Phytopathology, 79:361-66 (1989); Ristaino et al., “Influence of Isolates of Gliocladium virens and Delivery Systems on Biological Control of Southern Blight on Carrot and Tomato in the Field,” Plant Dis., 78:153-56 (1994); Ristaino et al., “Soil Solarization and Gliocladium virens Reduce the Incidence of Southern Blight in Bell Pepper in the Field,” Phytopathology, 84:1114 (1994)). Some products even control dissimilar pathogens. Deny controls Rhizoctonia, Pythium, Fusarium, as well as several nematodes. BlightBan A506 can be sprayed onto trees, strawberry, tomato, and potato plants to prevent frost damage and fire blight caused by Erwinia amylovora. Trichoderma spp. can control a wide variety of pathogens and appear in more products than any other microbe (Anti-Fungus; Binab T; Supresivit; T-22G and T-22HB; Trichopel, Trichoject, Trichodowels, and Trichoseal; TY). Products containing Trichoderma spp. control species of Amillaria, Botrytis, Chondrostrenum, Colletotrichum, Fulvia, Fusarium, Monilia, Nectria, Phytophthora, Plasmopara, Pseudoperonospora, Pythium, Rhizoctonia, Rhizopus, Sclerotinia, Sclerotium, Verticillium, and wood rot fungi.

Many biocontrol products are applied in agricultural environments of low ecological diversity in order to facilitate establishment of the biocontrol agent. For example, SoilGard and T-22G are mixed with soil-less potting mix. Similarly, Anti-Fungus is applied to soil following steaming or fumigation. Other biocontrol agents are used to protect plant parts. Galltrol-A, Nogall, Diegall, and Norbac 84C are all applied as root dips at transplant to prevent crown gall. Aspire, Bio-Save 10, and Bio-Save 11 are applied post-harvest to citrus or pome fruits to protect these fruits from post-harvest diseases. Several biocontrol agents, including Blue Circle, Epic, Kodiak, and T-22HB, are applied as seed treatments. Binab is applied by spraying, mixing with soilless potting mix, painting on surfaces or inserting pellets into wood to control rot in wood and wood products. Mycostop is applied as a spray, drench, or through irrigation.

In order for biocontrol to be a useful component of an integrated pest management system, research is needed in several critical areas. This integrated approach will rely on accurate assessments of populations of pathogens present in an agricultural field and knowledge of economic thresholds for pathogen damage. Research needs to be aimed at an understanding of ecological parameters important for crop production and survival and efficacy of biocontrol agents, and at identifying and developing new biocontrol agents for control of plant diseases. Knowledge of the biology and ecology of the biocontrol agent, pathogen, and host plant can help to exploit strengths or weaknesses of these organisms to improve control performance. Similarly, knowledge of the ecological, biological, and physical conditions needed for successful biocontrol will permit optimization of these conditions to achieve the best possible levels of control.

The influence of the host plant on the composition and size of microbial communities has received little attention thus far. Larkin and coworkers (Larkin et al., “Ecology of Fusarium oxysporum f. sp. Niveum in Soils Suppressive and Conducive to Fusarium Wilt of Watermelon,” Phytopathology, 83:1105-16 (1993); Larkin et al., “Effect of Successive Watermelon Plantings on Fusarium oxysporum and other Microorganisms in Soils Suppressive and Conducive to Fusarium Wilt of Watermelon,” Pathology, (1993)) reported a cultivar-specific rhizosphere effect on soil and rhizosphere microbial communities associated with different watermelon cultivars. One cultivar in particular, Crimson Sweet, promoted the development of microorganisms antagonistic to the Fusarium wilt pathogen. More research is needed to determine the role of this type of interaction in the enhancement of biocontrol.

One barrier to acquiring an understanding of soil microbial systems has been the lack of suitable techniques for assaying soil samples. Population sizes of many soil microbes, especially fungi, are difficult to measure accurately for several reasons. The term “colony forming unit” reflects the fact that colonies arising on a plate may have come from, for example, microconidia, macroconidia, chlamydospores, ascospores, hyphal fragments, or other propagules. Further, the efficiency of recovery of propagules may differ from one soil to the next. In some cases, such as with Fusarium spp., the pathogens cannot be distinguished morphologically from the nonpathogens. In addition, many microbes are not easily cultured on standard media, although they may play significant roles in disease suppression, as with the mycoparsite Sporidesmium sclerotivorum for control of lettuce drop (Adams et al., “Economical Biological Control of Sclerotinia Lettuce Drop by Sporidesmium sclerotivorum,” Phytopathology, 80:1120-24 (1990)). Finally, even when propagule numbers can be accurately estimated, the effectiveness of these propagules is dependent on their nutritional status and on the types and population sizes of other microbes present in the soil system. All of these shortcomings are compounded by the difficulty of sampling, particularly sampling of microsites. Research is needed to develop rapid, reliable, precise techniques for assaying soil microbial communities.

In the future, research should emphasize combinations of two or more biocontrol agents, since combinations may provide more consistent or more efficient control than a single biocontrol agent. For example, biocontrol agents with different optimal environmental conditions, or biocontrol agents with different mechanisms of action could be combined. Biocontrol agents may even act synergistically such as the combination of Fusarium oxysporum with Pseudomonas spp. to control Fusarium wilt (Lemanceau et al., “Biological Control of Fusarium Diseases by Fluorescent Pseudomonas and Non-pathogenic Fusarium,” Crop Prot., 10:279-86 (1991)). Research is also needed on combining biocontrol agents with other control methods. For example, sublethal heat (solarization) or pesticide stress may weaken a pathogen, making it more vulnerable to the action of biocontrol agents (Lifshitz et al., “The Effect of Sublethal Heating on Sclerotia of Sclerotium rolfsii,” Can. J. Microbiol., 29:1607-10 (1983); Tjamos, et al., “Detrimental Effects of Sublethal Heating and Talaromyces flavus on Microsclerotia of Verticillium dahliae,” Phytopathology, 85:388-92 (1995)). Suitable systems also need to be developed for production, formulation and delivery of biocontrol agents, because these processes can greatly affect efficacy of the biocontrol agent.

Despite the existence and use of biocontrol agents in agriculture, there continues to be a need for development of new plant biocontrol agents. The present invention is directed to fulfilling this need.

### SUMMARY OF THE INVENTION

The present invention is directed to isolated Paenibacillus macerans, Pseudomonas putida, and Sporobolomyces roseus which are useful as biocontrol agents.

The biocontrol agents are useful in a method of imparting to plants protection against plant pathogens. This method involves applying the biocontrol agent to plants, plant seeds, or soil surrounding plants under conditions effective to impart disease protection to plants or plants produced from the plant seeds.

The present invention is also directed to a method of enhancing plant growth. This involves applying the biocontrol agent to plants, plants seeds, or soil surrounding plants under conditions effective to enhance growth in the plants or plants produced from the plant seeds.

The biocontrol agents of the present invention are highly useful in agriculture to protect plants from a variety of plant bacterial, fungal, and viral diseases. In addition, these agents can enhance the growth of treated plants. Significantly, these effects are achieved without being hazardous to animals or humans.

### BRIEF DESCRIPTION OF THE INVENTION

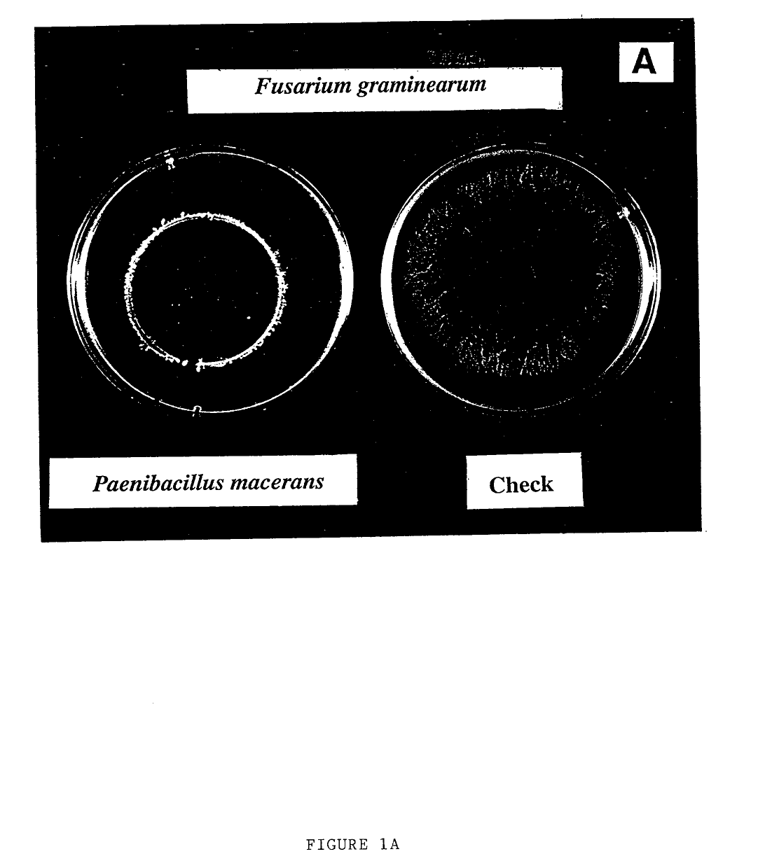

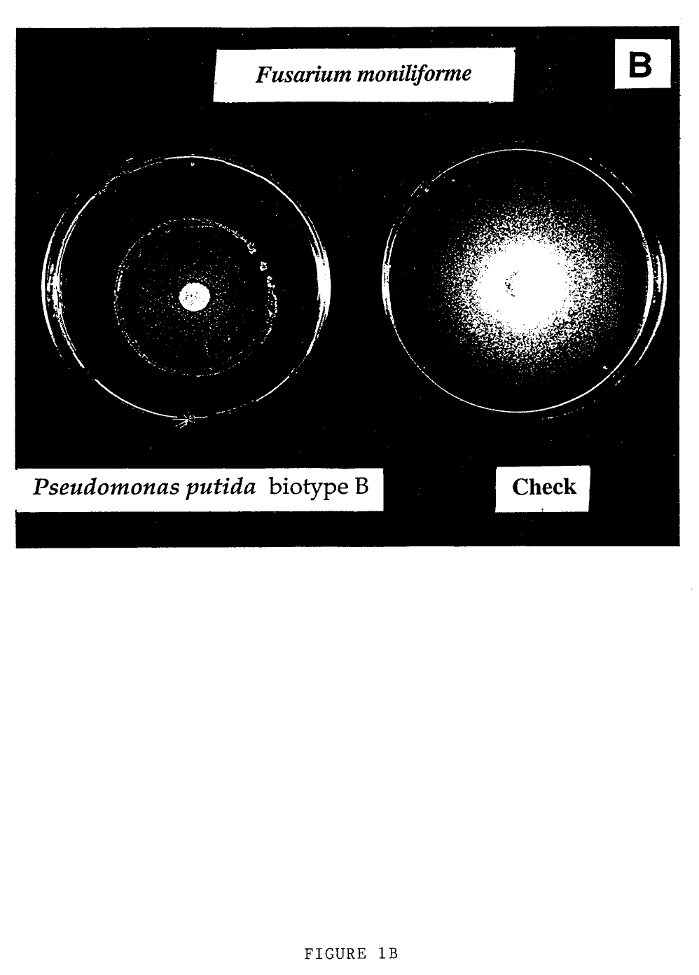

FIG. 1 shows paired in-vito assays for antibiosis of Fusarium graminearum and F. moniliforme by the following candidate bioprotectants: A) F. graminearum with and without Paenibacillus macerans; B) F. moniliforme with and without Pseudomonas putida; and C) F. graminearum with and without Sporobolomyces roseus.

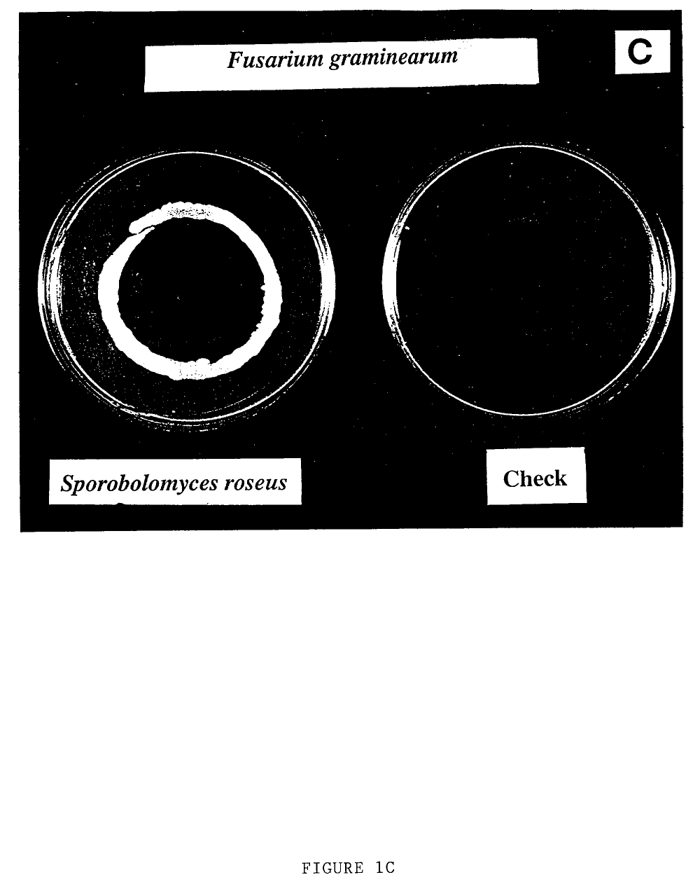

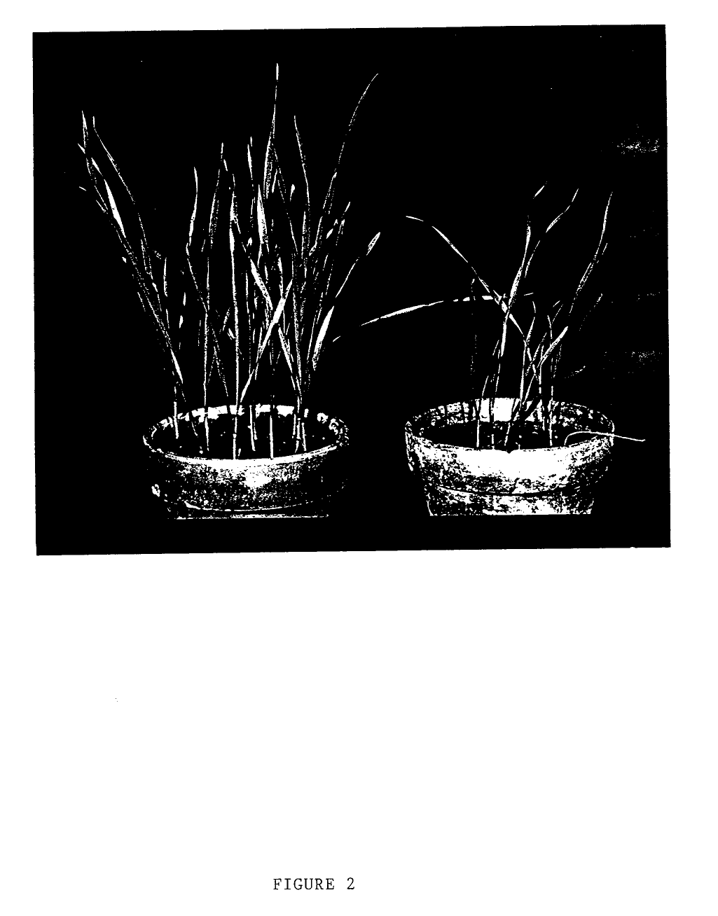

FIG. 2 shows wheat seeds naturally infected with F. graminearum. The plant on the right is grown from seed treated with Paenibacillus macerans, while the plant on the left is grown from nontreated seed.

### DETAILED DESCRIPTION OF THE INVENTION

The present invention is directed to isolated Paenibacillus macerans, Pseudomonas putida, and Sporobolomyces roseus, each of which are useful as biocontrol agent to impart disease protection to plants and to enhance plant growth.

The Paenibacillus macerans is a Gram variable rod, spore-forming bacteria. It is known as isolate 144 and has Embrapa Trigo Accession No. 144/88.4Lev, Cornell Accession No. Pma007BR-97, and ATCC Accession No. ______. This bacteria shows strong antibiosis against Cochliobolus sativus (spot blotch/common root rot of cereals), Colletotrichum graminicola (corn anthracnose), Fusarium graminearum (scab of cereals, ear/stalk rot of corn), Fusarium moniliforme (ear/stalk rot of corn), Pyrenophora tritici-repentis (tan spot of wheat), Stagonospora nodorum (Stagonospora nodorum blotch of wheat), Stagonospora avenae f. sp. triticea (Stagonospora avenae blotch of wheat), and Stenocarpella maydis (stalk/ear rot of corn). The Paenibacillus macerans of the present invention shows excellent control of seed borne transmission of Cochliobolus sativus, Pyrenophora tritici-riepentis, and Fusarium graminearum in wheat and of Fusarium moniliforme in corn. It also prevents aerial inoculation of flowering wheat spikes with Fusarium graminearum, diminishes grain infection frequency by Fusarium, reduces grain weight reduction by Fusarium, and dramatically reduces grain contamination by Fusarium mycotoxin deoxynivalenol. In addition, this bacterium may be used to reduce contamination of grains and other plant products with harmful secondary fungal metabolites. The endophytic capability of this bacterium suggests additional applications for plant disease control. Seedlings and other plant propagative units can be inoculated for long-term plant protection.

The Pseudomonas putida is a Gram positive rod, non-spore forming bacteria. It is known as biotype B isolate 63 and has Embrapa Trigo Accession No. 63/88 4 B, Cornell Accession No. Ppu002BR-97, and ATCC Accession No. ______. This bacteria shows strong antibiosis against Fusarium graminearum and some antibiosis against Cochliobolus sativus, Colletotrichum graminicola, Fusarium moiliforme, Stagonospora nodorum, and Stenocarpella maydis (stalk/ear rot of corn). The Pseudomonas putida of the present invention is effective in controlling seedborne transmission of Biopolaris sorokinianum and Fusarium graminearum in wheat and of Fusarium moniliforme in corn. It also shows excellent control of soilborne Fusarium graminearum in corn, activity against aerial inoculation of flowering wheat spikes with Fusarium, and strongly reduces grain contamination by the Fusarium mycotoxin deoxynivalenol.

The Sporobolomyces roseus is a red pigment yeast. It is known as isolate 53 and has Embrapa Trigo Accession No. 53/94.535, Cornell Accession No. Sro001BR-97, and ATCC Accession No. ______. This biocontrol agent is useful against aerial inoculation of flowering spikes with Fusarium graminearum, diminishes grain infection frequency by Fusarium, and reduces grain contamination by the Fusarium mycotoxin deoxynivalenol. This is a strongly competitive organism in colonizing organic substrates and is a profuse sporulater. It can suppress the survival and sporulation of debris-borne plant pathogens on crop residue and thereby reduce disease in a subsequently planted crop, especially under conservation tillage agriculture.

The biocontrol agents of the present invention are useful in a method of imparting to plants protection against plant pathogens. This method involves applying the biocontrol agent to plants, plant seeds, or soil surrounding plants under conditions effective to impart disease protection to the plants and to plants produced from the plant seeds.

The method of imparting pathogen protection to plants in accordance with the present invention is useful in protecting plants against a wide variety of pathogens including viruses, bacteria, and fungi. Plants can be protected against, inter alia, the following fungi by use of the method of the present invention: Fusarium oxysporum, Fusarium graminearum, Fusarium monilforme, Cochliobolus sativus, Collectotrichum graminicola, Stagonospora nodorum, Stagonospora avenae, Stenocarpella maydis, and Pyrenophora tritici-repentis.

The present invention is also directed to a method of enhancing plant growth by applying the biocontrol agents of the present invention to plants, plant seeds, and soil surrounding plants under conditions effective to enhance growth of the plants or plants resulting from the treated seeds.

With regard to the use of the biocontrol agents of the present invention to enhance plant growth, various forms of plant growth enhancement or promotion can be achieved. This can occur as early as when plant growth begins from seeds or later in the life of a plant. For example, plant growth according to the present invention encompasses greater yield, increased quantity of seeds produced, increased percentage of seeds germinated, increased plant size, greater biomass, more and bigger fruit, earlier fruit coloration, and earlier fruit and plant maturation. As a result, the present invention provides significant economic benefit to growers. For example, early germination and early maturation permit crops to be grown in areas where short growing seasons would otherwise preclude their growth in that locale. Increased percentage of seed germination results in improved crop stands and more efficient seed use. Greater yield, increased size, and enhanced biomass production allow greater revenue generation from a given plot of land.

The methods of the present invention can be utilized to treat a wide variety of plants or their seeds to impart disease protection and/or to enhance growth. Suitable plants include dicots and monocots. More particularly, useful crop plants can include: alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane. Examples of suitable ornamental plants are: Airabidopsis thaliana, Saintpaulia, petunia, pelargonium, poinsettia, chrysanthemum, carnation, and zinnia.

The methods of the present invention can be carried out through a variety of procedures when all or part of the plant is treated, including leaves, stems, roots, plant products (e.g., grain, fruit, forage, crop debris), propagules (e.g., cuttings), etc. Suitable application methods include high or low pressure spraying, drenching, and injection. When treating plant seeds, in accordance with the present invention, the biocontrol agent can be applied by low or high pressure spraying, coating, immersion, or injection. Biocontrol agents may also be applied to pathogen-infested crop stubble in order to reduce the inoculum available to infect a subsequent crop, especially under conservation tillage agriculture. Other suitable application procedures can be envisioned by those skilled in the art. Once treated with the biocontrol agents of the present invention, the seeds can be planted in natural or artificial soil and cultivated using conventional procedures to produce plants. After plants have been propagated from seeds treated in accordance with the present invention, the plants may be treated with one or more applications of the biocontrol agents of the present invention to impart disease protection to plants and/or to enhance plant growth.

The biocontrol agents can be applied to plants or plant seeds in accordance with the present invention alone or in a mixture with other materials. Alternatively, the biocontrol agent can be applied separately to plants with other materials being applied at different times.

A composition suitable for treating plants or plant seeds in accordance with the present invention contains a biocontrol agent in a carrier. Suitable carriers include water, aqueous solutions, slurries, solids (e.g., peat, wheat, bran, vermiculite, and pasteurized soil) or dry powders. In this embodiment, the composition contains 10⁶ to 10⁸, preferably 10⁷, colony forming units of the biocontrol agent per milliliter of carrier.

Although not required, this composition may contain additional additives including fertilizer, insecticide, fungicide, nematacide, and mixtures thereof. Suitable fertilizers include (NH₄)₂NO₃. An example of a suitable insecticide is Malathion. Useful fungicides include Captan.

Other suitable additives include buffering agents, wetting agents, coating agents, and abrading agents. These materials can be used to facilitate the process of the present invention. In addition, the biocontrol agent can be applied to plant seeds with other conventional seed formulation and treatment materials, including clays and polysaccharides.

### EXAMPLES

### Example 1

In-vitro Assays for Antibiosis of F. graminearum by Bioprotectants

In paired treatments, the radial growth (mm) of F. graminearum in the presence or absence of Paenibacillus macerans, Pseudomonas putida, or Sporobolomyces roseus was measured as a means of ascertaining the antibiosis of the pathogen by candidate bioprotectants. Each bacterial or yeast isolate was transferred onto 1/4 strength potato dextrose agar (i.e. PDA) in a circular pattern by means of a small sterile glass funnel. After 2 days of incubation at ambient temperature, an agar disk containing mycelia of the pathogen was transferred into the center of the ring-shaped colony of the bioprotectant or, in the control treatment, onto an uninoculated media plate. The radial growth of the pathogen was measured after 5 days of incubation. There was a minimum of four replicates per treatment.

### Example 2

Effect of Bioprotectants on the Infection of F. moniliforme-infested Maize Seed

Maize seed naturally infected with F. moniliforme and treated with Paenibacillus macerans and Pseudomonas putida were assayed for pathogen recovery. One hundred seeds were placed on culture dishes filled with 25% PDA (10 seeds/dish). The plates were rated for the presence of the pathogen after incubation for 5 days under fluorescent lights at ambient temperature. There were four replicates per treatment.

### Example 3

Effect of Bioprotectants on the Emergence of Maize Seed Planted in Soil Infested with F. graminearum

Maize seed treated with Paenibacillus macerans and Pseudomonas putida was planted in autoclaved greenhouse soil (Metro Mix) mixed 9:1 by volume with autoclaved, F. graminearum-inoculated oat kernels. There were four replicates of 100 seeds (10 seeds per pot) per treatment. All treatments were rated for % emergence 21 days after planting.

### Example 4

Effect of Seed-applied Bioprotectants on the Emergence of F. graminearum-infected Wheat Seeds

Wheat, cultivar NY Batavia, naturally infected with F. graminearum, was treated with slurries of Paenibacillus macerans and Pseudomonas putida and then planted in greenhouse soil (Metro Mix). There were four replicates of 100 seeds (10 seeds per pot) per treatment. All treatments were rated for % germination 21 days after planting.

### Example 5

Effect of Seed-applied Bioprotectants on Emergence of Wheat Planted in F. graminearum-infested Soil

Wheat seed treated with slurries of Paenibacillus macerans, Pseudomonas putida, or thiabendazole (Gustafson LSP 0.25 fl. oz. per 100 lb. seed) was planted in greenhouse soil (Metro Mix). There were four replicates per treatment. All treatments were rated for % germination 7 days after planting.

As shown below in Table 1, Paenibacillus macerans and P. putida, but not the S. roseus treatment, were shown to reduce significantly the radial growth of F. graminearum in vitro.

TABLE 1
Effect of Bioprotectants on the Growth of Fusarium
graminearum and F. moniliforme in Culture.

| | Mean Reduction in Radial Growth (mm) | |
|---|---|---|
| | Relative to the Nontreated Control | |
| Treatment | F. graminearum | F. moniliforme |
| Paenibacillus macerans | −13.4 | −20.5 |
| Pseudomonas putida | −15.2 | −6.5 |
| Sporobolomyces roseus | −3.4 | − |

As shown below in Table 2, all treatments reduced significantly the recovery of F. moniliforme from naturally infected maize seed. Paenibacillus macerans gave the greatest control. In this table, means within a column are significantly different (at P=0.05) from each other if they are followed by different letters, according to Duncan's multiple range test of significance. Little, et al., Agriculture Experimentation, p. 350 (1978), which is hereby incorporated by reference.

TABLE 2
Effect of Bioprotectants on the Growth of F. moniliforme
from Naturally Infested Maize Seed.

| Treatment | % Recovery on PDA |
|---|---|
| Non-treated | 35 a |
| Paenibacillus macerans | 13 d |
| Pseudomonas putida | 18 c |

As shown in Table 3, all treatments resulted in significantly greater emergence than the nontreated control. In this table, means within a column are significantly different (at P=0.05) from each other if they are followed by different letters, according to Duncan's multiple range test of significance.

TABLE 3
Effect of Seed-applied Bioprotectants on the Emergence
Maize Planted in F. graminearum-infested Soil.

| Treatment | Mean % emergence |
|---|---|
| Non-treated | 62 d |
| Paenibacillus macerans | 86 a |
| Pseudomonas putida | 83 ab |

Paenibacillus macerans and Pseudomonas putida treatments resulted in significantly greater emergence than the nontreated control, as shown in Table 4. In this table, means within a column are significantly different (at P=0.05) from each other if they are followed by different letters, according to Duncan's multiple range test of significance.

TABLE 4
Effect of Seed-applied Bioprotectants on the Emergence
F. graminearum-infected Wheat Seeds Planted in Soil.

| Treatment | % Emergence |
|---|---|
| Non-treated | 63 c |
| Paenibacillus macerans | 72 a |
| Pseudomonas putida | 69 ab |

As shown in Table 5, all treatments resulted in significantly greater emergence than the nontreated control. Paenibacillus gave the greatest % emergence after the thiabendazole treatment. In this table, means within a column are significantly different (at P=0.05) from each other if they are followed by different letters, according to Duncan's multiple range test of significance.

TABLE 5
Effect of Seed-applied Bioprotectants on the Emergence of
Wheat Seeds Planted in F. graminearum-infested Soil.

| Treatment | % Germination |
|---|---|
| Non-treated | 50 e |
| Paenibacillus macerans | 76 b |
| Pseudomonas putida | 69 d |
| Thiabendazole | 80 a |

All bioprotectants tested showed some control of seed rot and seedling blight caused by Fusarium graminearum or F. moniliforme. The spore-forming bacterium, Paenibacillus macerans, showed the greatest promise as a bioprotectant against seedborne and soilborne Fusarium species.

### Example 6

Bioprotection of Wheat

Seeds of wheat cultivar Embrapa 24 infected by Pyrenophora tritici-repentis were obtained from the basic seed production service, Embrapa, Passo Fundo, RS, Brazil. The following bacterial and fungal bioprotectants were applied to seed: Paenibacillus macerans and Pseudomonas putida biotype B. Iprodione plus thiram-treated and nontreated seeds were used as controls. Colonies of each bacterium were grown on 1/4 potato dextrose agar (i.e. PDA) for 24 hr at 24±2° C. Bacterial cells were removed from the surface of the culture medium with a brush and placed in sterile distilled water. The concentration of each bacterium was approximately 10⁶ CFU/ml. A suspension was then applied by dipping the seeds for 3 min., and allowing them to dry at room temperature for 24 hr. The fungicide mixture was tested at a dosage of 150 g Rovrin WP per 100 kg of seeds. Nontreated seeds were soaked in sterile distilled water, for 3 min, and allowed to dry in the same manner as microbial-treated seeds. For the laboratory experiment, each treatment was replicated four times (each replicate 100 grains, 10 grains per plate) and placed under black light under a photoperiod of 12 hr at 24±2° C. The experimental arrangement was a completely randomized design. The presence of P. tritici-repentis was determined 5 days after plating. The data were expressed as percentage of seeds from which the pathogen was recovered. An experiment was done to evaluate the incidence of transmission in a highly infected seed lot. Transmission was scored as the percentage of seedlings with characteristic coleoptile lesions.

Radial growth in the laboratory test was measured using the funnel method (Luz, W. C., “Controle biológico das doenças na espermosfera,” Controle biológico de doenças de plantas, EMBRAPA-CNPDA, Jaguari na, Brasil pages 25-31 (1991), which is hereby incorporated by reference).

For the field experiment, seeds with each treatment were manually sown in plots of 12 rows, 3 m long. The space between rows was 20 cm and the amount of seeds was equivalent to 120 kg per ha. Treated plots in each experiment were arranged in a randomized block design. Emergence was measured 21 days after sowing. At maturity, eight central rows of each plot were harvested and the yield was determined as kg/ha. The data were subjected to analysis of variance and the means separated by Duncan's multiple range test (P=0.05).

Both bioprotectant bacteria inhibited strongly the radial growth of P. tritici-repentis and its recovery from infected seed (Table 6). Treatments with Paenibacillus macerans and Pseudomonas putida biotype B, inhibited completely the transmission of P. tritici-repentis to seedlings (Table 6).

TABLE 6
Effect of Bioprotectants on Radial Growth, Recovery from
Infected Seeds, and Percent Seed Transmission of
Pyrenophora tritici-repentis.

| | Radial | % recovery | | % |
|---|---|---|---|---|
| | growth | from seeds | | transmission |
| Treatments | (cm) | lot I | lot II | lot I |
| Nontreated | 2.5 | 62 | 22 | 16 |
| Paenibacillus macerans | 0.0 | 0 | 0 | 0 |
| Pseudomonas putida biotype B | 0.2 | 0 | 0 | 0 |
| Iprodione + thiram | − | 0 | 0 | 0 |
| Rovrin WP | | | | |

Seeds were also naturally infected, but at a lower incidence, by Fusarium graminearum Schwabe and Bipolaris sorokinianum (Sacc.) Shoem.; the two bacteria also inhibited recovery of these pathogens from seed.

Data from the field experiment are shown in Table 7.

TABLE 7
Effect of Seed-applied Bioprotectants on Seedling Emergence
and Yield of Wheat in the Field

| | Seedling | Yield |
|---|---|---|
| Treatments | emergence | (kg/ha) |
| Nontreated | 311 c | 1666 c |
| Paenibacillus macerans | 326 a | 2201 a |
| Pseudomonas putida biotype B | 336 a | 2112 a |
| Iprodione + thiram | 330 a | 2110 a |
| Rovrin WP | | |

All biological or chemical treatments significantly increased seedling emergence of wheat over that in the nonprotected plots. Paenibacillus macerans and Psedomonas putida biotype B provided the greatest yield increase over that of nonprotected plots (Table 7).

The beneficial effects of plant growth promoting and bioprotecting rhizobacteria (i.e. PGPBR) on plants have been reviewed (Bakker et al., “Suppression of Soil-Borne Plant Pathogens by Fluorescent Pseudomonads: Mechanisms and Prospects,” Biotic Interactions and Soil Borne Diseases, Ed. A.B.R. Beemster, et al., pp. 217-23, Amsterdam, The Netherlands, Elsevier (1991); Kloepper, J. W. “Plant Growth-Promoting Rhizobacteria as Biological Control Agents of Soilborne Diseases,” The Biological Control of Plant Diseases, pp. 142-52 (1991); Luz, W. C., “Microbiolização de sementes para o controle de doenças das plantas,” Revisão Annual de Patologia de Plantas, Passo Fundo, Brasil, W. C. da Luz, J. M. C. Fernandes, A. M. Prestes and E. C. Picinini, ed., pages 35-77 (1993); Luz, W. C., “Rizobabactérias promotoras de crescimento de plantas e de bioproteção,” Revisão Annual de Patologia de Plantas, Passo Fundo, Brasil, W. C. da Luz, J. M. C. Fernandes, A. M. Prestes and E. C. Picinini, ed., pages 1-49 (1996), which are hereby incorporated by reference), as having the benefits of fungal bioprotectants (Harman, G. E., “Seed Treatments for Biological Control of Plant Disease,” Crop. Prot., 10:166-71 (1991), which is hereby incorporated by reference). Microbial agents used as bioprotectants and yield stimulants will be a significant tactic of disease management in the next century (Luz, W. C. da RizababactÈrias promotoras de crescimento de plantas e de bioproteÁ,,o. Pages 1-49 in W. C. da Luz, J. M. C. Fernandes, A. M. Prestes and E. C. Picinini, ed., Revis,, o Anual de Patologia de Plantas., Passo Fundo, Brasi (1996), which are hereby incorporated by reference). The two PGPBRs reported here show great promise as yield stimulators and bioprotectants of wheat against diseases.

### Example 7

Biocontrol of Wheat Scab with Microbial Antagonists

Plants of spring wheat ND594 were grown in a glasshouse at Cornell University, Ithaca, N.Y. At mid-anthesis stage, spikes of plants were either sprayed with water or with cell suspensions of potential bioprotectant microorganisms. All spikes were challenge-inoculated 24 hr later with a F. graminearum spore suspension, and the plants were incubated overnight at high relative humidity. Thereafter, the plants were allowed to grow on a glasshouse bench under ambient conditions through grain maturation. Harvested spikes were evaluated for incidence of seeds infected by Fusarium, 100-kernel weight, and deoxynivalenol (DON) content. Fusarium infection was determined by characteristic growth of the pathogen from seed incubated on blotters after commencement of germination followed by freezing to kill the seed embryos. DON was analyzed by high pressure liquid chromatagraphy at the Cornell Veterinary Diagnostic Laboratory.

Paenibacillus macerans (Embrapa-Trigo), Pseudomonas putida biotype B (Embrapa-Trigo), and Sporobolomyces roseus (Embrapa-Trigo) consistently protected spikes and resulted in average (three experiments) increases in kernel weight over those of nonprotected spikes. Table 8 shows the protection of wheat grains by microbial strains against weight reduction, Fusarium infection, and contamination by the Fusarium mycotoxin, deoxynivalenol. (Means of four replicates with standard errors in parentheses; results of one of three experiments shown).

TABLE 8

| | | | | Deoxynivalenol |
|---|---|---|---|---|
| | | Grain weight | Incidence of | contamination |
| Inoculation 1 | Inoculation 2 | (g/100 seeds) | Fusarium (%) | (ppm) |
| Water | Water | 2.86 (0.37) | 0 (0.0) | 0.16 (0.16) |
| Water | Fusarium | 2.06 (0.54) | 34 (6.2) | 10.70 (8.02) |
| Sporobolomyces roseus | Fusarium | 2.08 (0.53) | 25 (12.8) | 4.08 (2.73) |
| Pseudomona putida | Fusarium | 2.52 (0.54) | 17 (8.5) | 1.55 (1.23) |
| Paenibacillus macerans | Fusarium | 2.75 (0.14) | 15 (14.2) | 0.63 (0.86) |

### Example 8

Inhibition of Mycelial Growth (Antibiosis) of Fungal Pathogens of Cereals by Paenibacillus macerans Isolate 144 from Brazil

One indication that a microorganism strain may be useful for biocontrol of fungal plant pathogens is its ability to inhibit mycelial (filamentous) growth of fungi in vitro. Paenibacillus macerans isolate 144, a spore-forming bacterium isolated from roots of wheat plants in Brazil, was tested for antibiosis in vitro against a broad range of economically important fungal pathogens of wheat and corn.

In paired treatments, in the presence or absence of P. macerans isolate 144, the radial growth (mm) of fungal pathogens was measured as a means of estimating antibiosis. Bacterial cells were transferred onto a Petri dish of 1/4 strength potato dextrose agar by means of a small glass funnel. After two days incubation at room temperature, an agar disk containing mycelia of the fungus was transferred into the center of the ring-shaped colony of the bioprotectant or, in the nontreated, onto an uninoculated media plate. The radial growth was measured after 5 days. There were a minimum of four replicates per experiment.

Paenibacillus macerans isolate 144 strongly inhibited the mycelial growth of several cereal pathogens in vitro (Table 9). This indicates the production of antibiotic(s) active against a broad range of plant pathogenic fungi. Antibiosis is a useful trait for a plant disease biocontrol strain. The antibiotic(s) may also find direct uses as antimycotics in agriculture or medicine.

TABLE 9
In vitro Inhibition by Paenibacillus macerans Isolate 144 of Mycelial
Growth of Fungi Pathogenic to Cereals

| | Radial Growth of Mycelium (mm) | |
|---|---|---|
| Plant pathogenic fungus: | Fungus | Fungus in presence of |
| (plant disease) | alone | P. macerans |
| Cochliobolus sativus | 23 a* | 2 b |
| (spot blotch of wheat) | | |
| Pyrenophora tritici-repentis | 25 a | 0.5 b |
| (tan spot of wheat) | | |
| Stagonospora nodorum | 5 a | 1 b |
| (S. nodorum blotch of wheat) | | |
| Stagonospora avenae | 12 a | 1 b |
| f. sp. triticea (S. avenae | | |
| blotch of wheat | | |
| Colletotrichum graminicola | 25 a | 2 b |
| (anthracnose of corn) | | |
| Stenocarpella maydis | 21 a | 2 b |
| (Stenocarpella ear and stalk | | |
| rot of corn) | | |
| Stenocarpela macrospora | 40 a | 5 b |
| (Stenocarpella ear and stalk | | |
| rot of corn) | | |
| Fusarium moniliformne | 36 a | 7 b |
| (Fusarium ear and stalk | | |
| rot of corn) | | |
| Fusarium graminearum | 24 a | 11 b |
| (wheat scab; and Gibberella | 14 a | 7 b |
| ear and stalk rot of corn) | | |

*Means within a row (same fungus) and following by different letters differ significantly according to the protected Duncan's multiple range test.

### Example 9

Control of Seedborne Cereal Diseases by Seed Treatment with Microbial Antagonists

Microbial bioprotectants may provide a safe and effective alternative or complement to chemical fungicides for control of seedborne fungal pathogens in cereals and other crop plants.

Seed lots of wheat and corn naturally infected by pathogenic fungi were located and used for testing the efficacy of seed-applied bioprotectants. Seeds were either soaked in water or cell suspensions of Paenibacillus macerans isolate 144 or Pseudomonas putida biotype B isolate 63, then allowed to dry briefly before incubation on moist blotter papers (for isolation of fungi) or sowing in soil. Seedlings were scored for percentage emergence and pathogen transmission (based on coleoptile lesions).

Paenibacillus macerans isolate 144 and Pseudomonas putida biotype B isolate 63 applied to fungal-infected seeds of corn and wheat each resulted in significantly less seedborne inoculum and less seed-to-seedling disease transmission of several fungal pathogens (Table 10). They also resulted in increased emergence of infected seedlings in soil. Both show considerable potential as biological seed protectants for cereal seeds.

TABLE 10
Biocontrol of Seedborne Fungal Pathogens of Cereals as Measured
by Decreased Recovery of Fungi from Seed, Decreased
Transmission of Fungi to Seedlings, and Increased
Seedling Emergence.

| | Seed treatment | | |
|---|---|---|---|
| | | Paenibacillus | Pseudomonas |
| Seedborne fungus: | Nontreated | macerans 144 | putida 63 |
| | Isolation from seeds (%) | | |
| Cochliobolus sativus | 18 a* | 0 c | 2 b |
| (wheat seed) | | | |
| Fusarium moniliforme | 35 a | 13 c | 18 b |
| (corn seed) | | | |
| | Transmission to seedlings (%) | | |
| Cochliobolus sativus | 46 a | 7 c | 12 b |
| (wheat seed in field | | | |
| soil) | | | |
| Cochliobolus sativus | 57 a | 10 c | 17 b |
| (wheat seed in | | | |
| autoclaved soil) | | | |
| Stagonospora nodorum | 47 a | 15 c | 11 b |
| (wheat seed in soil) | | | |
| | Emergence of seedlings (%) | | |
| Cochliobolus sativus | 54 b | 70 a | 56 b |
| (wheat seed in soil) | | | |
| Fusarium | 50 c | 76 a | 69 b |
| graminearum (wheat | | | |
| seed in soil) | | | |
| Stagonospora nodorum | 88 c | 94 a | 91 b |
| (wheat seed in soil) | | | |

*Means within a row (same seedlot) and followed by different letters differ significantly according to the protected Duncan's multiple range test.

Although the invention has been described in detail for the purpose of illustration, it is understood that such detail is solely for that purpose, and variations can be made therein by those skilled in the art without departing from the spirit and scope of the invention which is defined by the following claims.

What is claimed:

1. An isolated Paenibacillus macerans having ATCC Accession No. ______.

2. An isolated Pseudomonas putida having ATCC Accession No. ______.

3. An isolated Sporobolomyces roseus having ATCC Accession No. ______.

4. A composition comprising:
an isolated Paenibacillus macerans according to claim 1 and a carrier.

5. A composition according to claim 4 further comprising a fertilizer, insecticide, fungicide, nematacide, or mixtures thereof.

6. A composition according to claim 4 further comprising buffering agents, wetting agents, coating agents, abrading agents, clay, polysaccharides, or mixtures thereof.

7. A composition comprising:
an isolated Pseudomonas putida according to claim 2 and a carrier.

8. A composition according to claim 7 further comprising a fertilizer, insecticide, fungicide, nematacide, or mixtures thereof.

9. A composition according to claim 7 further comprising buffering agents, wetting agents, coating agents, abrading agents, clay, polysaccharides, or mixtures thereof.

10. A composition comprising:
an isolated Sporobolomyces roseus according to claim 3 and a carrier.

11. A composition according to claim 10 further comprising a fertilizer, insecticide, fungicide, nematacide, or mixtures thereof.

12. A composition according to claim 10 further comprising buffering agents, wetting agents, coating agents, abrading agents, clay, polysaccharides, or mixtures thereof.

13. A method of imparting to plants protection against plant pathogens comprising:
applying to plants, plants seeds, or soil surrounding plants or plant seeds a biocontrol agent selected from the group consisting of Paenibacillus macerans, Pseudomonas putida, Sporobolomyces roseus, and mixtures thereof under conditions effective to impart disease protection to the plants or plants produced from the plant seeds.

14. A method according to claim 13, wherein the biocontrol agent is Paenibacillus macerans having ATCC Accession No. ______.

15. A method according to claim 13, wherein the biocontrol agent is Pseudomonas putida having ATCC Accession No. ______.

16. A method according to claim 13, wherein the biocontrol agent is Sporobolomyces roseus having ATCC Accession No. ______.

17. A method according to claim 13, wherein the biocontrol agent is used to treat plants by topical application.

18. A method according to claim 13, wherein the biocontrol agent is used to treat plant seeds by topical application, said method further comprising:
propagating plants from said seeds topically treated with the biocontrol agent.

19. A method according to claim 13, wherein the biocontrol agent is used to treat soil around the plants.

20. A method according to claim 13, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, and sugarcane.

21. A method according to claim 13, wherein the plant is selected from the group consisting of Arabidopsis thaliana, Saintpaulia, petunia, pelargonium, poinsettia, chrysanthemum, carnation, and zinnia.

22. A method of enhancing plant growth comprising:
applying to plants, plants seeds, or soil surrounding plants a biocontrol agent selected from the group consisting of Paenibacillus macerans, Pseudomonas putida, Sporobolomyces roseus, and mixtures thereof under conditions effective to enhance growth in the plants or plants produced from the plant seeds.

23. A method according to claim 22, wherein the biocontrol agent is Paenibacillus macerans having ATCC Accession No. ______.

24. A method according to claim 22, wherein the biocontrol agent is Pseudomonas putida having ATCC Accession No. ______.

25. A method according to claim 22, wherein the biocontrol agent is Sporobolomyces roseus having ATCC Accession No. ______.

26. A method according to claim 22, wherein the biocontrol agent is used to treat plants by topical application.

27. A method according to claim 22, wherein the biocontrol agent is used to treat plant seeds by topical application, said method further comprising:
propagating plants from said seeds topically treated with the biocontrol agent.

28. A method according to claim 22, wherein the biocontrol agent is used to treat soil around the plants.

29. A method according to claim 22, wherein the plant is selected from the group consisting of alfalfa, rice, wheat, barley, rye, cotton, sunflower, peanut, corn, potato, sweet potato, bean, pea, chicory, lettuce, endive, cabbage, brussel sprout, beet, parsnip, turnip, cauliflower, broccoli, turnip, radish, spinach, onion, garlic, eggplant, pepper, celery, carrot, squash, pumpkin, zucchini, cucumber, apple, pear, melon, citrus, strawberry, grape, raspberry, pineapple, soybean, tobacco, tomato, sorghum, sugarcane.

30. A method according to claim 22, wherein the plant is selected from the group consisting of Arabidopsis thaliana, Saintpaulia, petunia, pelargonium, poinsettia, chrysanthemum, carnation, and zinnia.
